(19) United States
(12) Patent Application Publication
(10) Pub. No.: US 2021/0079100 A1
(43) Pub. Date: Mar. 18, 2021

(54) METHODS AND COMPOSITIONS FOR TREATING HYPERPIGMENTATION DISORDERS

(71) Applicants: INSERM (Institute National de la Sante et de la Recherche Medicale), Paris (FR); Universite Nice Sophia Antipolis, Nice (FR); Centre Hopitalier Universitaire de Nice, Nice (FR)

(72) Inventors: Thierry PASSERON, Nice Cedex 3 (FR); Laura SORMANI LE BOURHIS, Nice (FR)

(21) Appl. No.: 16/620,180

(22) PCT Filed: Jun. 7, 2018
(86) PCT No.: PCT/EP2018/065082

§ 371 Date: Dec. 6, 2019

(30) Foreign Application Priority Data
Jun. 8, 2017 (EP) 17305681.3

(51) Int. Cl. C07K 16/28; C12N 15/113; A61K 31/7105; A61P 17/00

(52) U.S. Cl. CPC C07K 16/2851; A61P 17/00; A61K 31/7105; C12N 15/1138

(57) ABSTRACT

The present invention relates to a method for treating hyperpigmentary skin disorder. By using normal human melanocytes (NHMs) and normal human keratinocytes (NHKs), which are infected with CLEC12B siRNA/shRNA/RNAi lentiviral particles, inventors have showed that decreasing CLEC12B expression significantly reduce the transfer of melanin to the keratinocytes. These results demonstrate that CLEC12B is specifically expressed in the skin by melanocytes and plays a key role in the transfer or melanosomes to the keratinocytes. Accordingly, the invention relates to a method for treating hyperpigmentary skin disorder in a subject in need thereof comprising a step of administering to said subject a therapeutically effective amount of a CLEC12B antagonist, wherein CLEC12B antagonist is polypeptide, more particularly a decoy.

### FIELD OF THE INVENTION

The invention is in the field of dermatology, more particularly the invention relates to methods and compositions for treating hyperpigmentation disorders.

### BACKGROUND OF THE INVENTION

Pigmentation disorders are disturbances of human skin color, either loss or reduction (depigmentation or hypopigmentation) which may be related to loss of melanocytes or to the inability of melanocytes to produce melanin or transport melanosomes correctly, or increase (hyperpigmentation) which is caused by an excessive production of melanin by melanocytes. Melanocytes are located at the lower layer (the stratum basale) of the skin's epidermis, the middle layer of the eye (the uvea), the inner ear, meninges, bones, and heart. Melanin is a blackish-brown pigment produced by melanocytes and is an insoluble high molecular weight compound formed by an oxidative condensation reaction by the action of enzymes such as tyrosinase using the amino acid, tyrosine, as a substrate. More specifically, melanin is classified into eumelanins, which exhibit black color, and pheomelanins, which exhibit brown-red color. After being produced by melanocytes, these melanins are transferred in the form of melanin-containing granules (melanosomes) to epidermal keratinocytes present in the periphery thereof, and are excreted from epidermis accompanying turnover thereof. 1-2% of population are affected by hypopigmentation (e.g vitiligo) and up to 40% of woman in some countries are affected by hyperpigmentation (e.g melasma). These pigmentation disorders alter people's quality of life, the effectiveness of treatments is limited resulting in unsatisfactory outcomes, and there is a high therapeutic demand. Thus there is a need to identify new treatments against pigmentation disorders.

### SUMMARY OF THE INVENTION

The invention relates to methods and compositions for treating hyperpigmentation disorders. In particular, the present invention is defined by claims.

### DETAILED DESCRIPTION OF THE INVENTION

The inventors have performed transcriptional analysis on lesional, perilesional and non-depigmented skin from vitiligo patients and on matched skin from healthy subject. They have observed that the expression level of CLEC12B is correlated with key proteins involved in the pigmentation. For the first time, the inventors have demonstrated that CLEC12B has a specific expression in melanocytes. More particularly, they have showed that decreasing CLEC12B expression, or impairing CLEC12B binding to keratinocytes, significantly reduce the transfer of melanin to the keratinocytes. Thus, the inventors have found a new target suitable for the treatments of hyperpigmentation disorders.

### Method for Treating Hyperpigmentation Disorders

Accordingly, the invention relates to a method of treating hyperpigmentary skin disorder in a subject in need thereof comprising a step of administering to said subject a therapeutically effective amount of a CLEC12B antagonist.

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the terms “hyperpigmentary skin disorder” or “hyperpigmentation disorder” are used interchangeably and refer to the darkening of an area of skin or nails caused by increased melanin. Hyperpigmentation is the result of either of two occurrences: (1) an abnormally high concentration of melanocytes produce melanin or (2) when melanocytes are hyperactive. Hyperpigmentation disorders are can affect any part of the body including the face, hands, and neck. Hyperpigmentation disorder is selected form the group consisting of but not limited to solar lentigines, melasma, freckles, age spots, post-acne pigmentation and post-inflammatory hyperpigmentation. The term “lentigo/lentigenes” or “solar lentigines,” also known as a sun-induced freckle or senile lentigo, is a dark (hyperpigmented) lesion caused by natural or artificial ultraviolet (UV) light. The term “melasma” also called as pregnancy-induced melasma. It is also known as pregnancy mask or chloasma. With melasma, the pigmentation is generally symmetrical and has clearly defined edges. The term “freckles” refers to flat circular spots which are usually tan or light brown in colour. While freckles are an extremely common type of hyperpigmentation, they are more often seen among people with a lighter skin tone. The term “age spots” refers to tan, brown or black in colour. Age spots are oval in shape and the size varies from freckle size to more than 13 mm. It is also known as liver spots and they tend to develop on the face and other photo-exposed areas after the age of 40. The term “post acne pigmentation” refers to marks caused by acne. They can be observed in more than 60% of acne in some ethnies. In most cases pigmentary marks which are dark in colour result from an overproduction of melanin in reaction to skin inflammation at the affected area. Without proper treatment, post-acne pigmentation may take months or even years to fade off. The term “post inflammatory hyperpigmentation” refers to the marks caused by an injury or inflammation to the skin, there is an increased production of colour pigment in such conditions.

As used herein, the term “subject” refers to any mammals, such as a rodent, a feline, a canine, and a primate. Particularly, in the present invention, the subject is a human. More particularly, the subject is a human suffering from one of the hyperpigmentation disorders as describes above.

As used herein, the term “CLEC12B”, refers to C-type lectin domain family 12 member B considered as a transmembrane receptor. It is a protein that in humans is encoded by the CLEC12B gene. The naturally occurring human CLEC12B gene has a nucleotide sequence as shown in Genbank Accession number NM_001129998.2 and the naturally occurring human ID3 protein has an aminoacid sequence as shown in Genbank Accession number NP_001123470.1. The murine nucleotide and amino acid sequences have also been described (Genbank Accession numbers NM_001204223.1 and NP_001191152.1). C-type lectin is a family of transmembrane receptors that bind carbohydrates via a carbohydrate recognition domain. CLEC12B possess an ITIM domain which can recruit phosphatases like SHP-1 or SHP-2 upon phosphorylation. SHP-1 and SHP-2 are able to de-phosphorylate and to inhibit intracellular factors such as STAT1 and STAT3.

As used herein, the term “antagonist” has its general meaning in the art, and refers to a compound which has the capability of reducing or suppressing selectively the activity or expression of CLEC12B. In the context of the invention, the compound inhibits the interaction of CLEC12B with keratinocytes. CLEC12B interacts with glycoproteins which are present on keratinocytes. Typically, glycoproteins contain oligosaccharide chains (glycans) covalently attached to amino acid side-chains. Example of glycoproteins well known in the art: β-D-Galactose, β-D-Glucose, D-Mannose, α-L-Fucose, N-Acetylgalactosamine etc This inhibition of interaction allows to reduce the transfer of melanosomes from melanocytes to keratinocytes. Melanosomes contain melanin which is responsible of the pigmentation. Thus, the antagonist of CLEC12B reduces the transfer of melanin from melanosomes to keratinocytes.

In some embodiments, the CLEC12B antagonist is a small organic molecule, an antibody, a polypeptide, an aptamer, a siRNA or an oligonucleotide. In a particular embodiment, the CLEC12B antagonist is a small molecule. The term “small organic molecule” as used herein, refers to a molecule of a size comparable to those organic molecules generally used in pharmaceuticals. The term excludes biological macro molecules (e.g. proteins, nucleic acids, etc.). Typically, small organic molecules range in size up to about 5000 Da, more preferably up to 2000 Da, and most preferably up to about 1000 Da. In some embodiments, the CLEC12B antagonist is a glycomimetic molecule. As used herein, the term “glycomimectic molecule” refers to compounds that mimic the bioactive function of carbohydrates and address the drawbacks of carbohydrate leads, namely their low activity and insufficient drug-like properties. Typically, the glycomimectic molecule is selected from the group consisting of: Cylexin (CY-1503), Bimosiamose (TBC-1269), OJ-R9188, GMI-1070, PSI-697, GSC-150, Efomycin M, as described in Ernst et al 2009, Nature Reviews Drug Discovery. Such glycomimetic molecules are useful for the blocking of CLEC12B interaction with its ligands and thus blocking the transfer of melanosomes from melanocytes to keratinocytes.

In a particular embodiment, the CLEC12B antagonist is an antibody. The term “antibody” is thus used to refer to any antibody-like molecule that has an antigen binding region, and this term includes antibody fragments that comprise an antigen binding domain such as Fab′, Fab, F(ab′)2, single domain antibodies (DABs or VHH), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art.

In some embodiments, the antibody is a monoclonal antibody. Monoclonal antibodies can be prepared and isolated using any technique that provides for the production of antibody molecules by continuous cell lines in culture. Techniques for production and isolation include but are not limited to the hybridoma technique, the human B-cell hybridoma technique and the EBV-hybridoma technique.

In some embodiments, the antibody is non-internalizing. As used herein the term “non-internalizing antibody” refers to an antibody, respectively, that has the property of to bind to a target antigen present on a cell surface, and that, when bound to its target antigen, does not enter the cell and become degraded in the lysosome.

Particularly, in the context of the invention, the antibody is a single domain antibody. The term “single domain antibody” has its general meaning in the art and refers to the single heavy chain variable domain of antibodies of the type that can be found in Camelid mammals which are naturally devoid of light chains. Such single domain antibody are also called VHH or “nanobody®”. For a general description of (single) domain antibodies, reference is also made to the prior art cited above, as well as to EP 0 368 684, Ward et al. (Nature 1989 Oct 12; 341 (6242): 544-6), Holt et al., Trends Biotechnol., 2003, 21(11):484-490; and WO 06/030220, WO 06/003388. In the context of the invention, the amino acid residues of the single domain antibody are numbered according to the general numbering for VH domains given by the International ImMunoGeneTics information system aminoacid numbering (http://imgt.cines.fr/). Particularly, in the context of the invention, the antibody is a single chain variable fragment. The term “single chain variable fragment” or “scFv fragment” refers to a single folded polypeptide comprising the VH and VL domains of an antibody linked through a linker molecule. In such a scFv fragment, the VH and VL domains can be either in the VH-linker-VL or VL-linker-VH order. In addition to facilitate its production, a scFv fragment may contain a tag molecule linked to the scFv via a spacer. A scFv fragment thus comprises the VH and VL domains implicated into antigen recognizing but not the immunogenic constant domains of corresponding antibody.

In a particular embodiment, the CLEC12B antagonist is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity.

In a particular embodiment, the CLEC12B antagonist is a polypeptide. The term “polypeptide” refers both short peptides with a length of at least two amino acid residues and at most 10 amino acid residues, oligopeptides (11-100 amino acid residues), and longer peptides (the usual interpretation of “polypeptide”, i.e. more than 100 amino acid residues in length) as well as proteins (the functional entity comprising at least one peptide, oligopeptide, or polypeptide which may be chemically modified by being glycosylated, by being lipidated, or by comprising prosthetic groups). In a particular embodiment, the polypeptide is a functional equivalent fragment of CELC12B. As used herein, a “functional equivalent” also known as a decoy or “decoy receptor”, as “sink” or “trap” is a compound which is capable of binding to soluble CLEC12B, thereby preventing its interaction with CLEC12B receptor. More particularly, it is a compound that binds to a ligand, but is structurally incapable of signaling or presenting the agonist to signaling receptor complexes. A decoy acts as a molecular trap for the ligand, thereby preventing it from binding to its functional receptor. A decoy is preferably soluble. A decoy can be a CLEC12B polypeptide or a fragment thereof. The term “functionally equivalent fragment” thus includes any equivalent of CLEC12B obtained by altering the amino acid sequence, for example by one or more amino acid deletions, substitutions or additions such that the protein analogue retains the ability to bind to soluble CLEC12B. Amino acid substitutions may be made, for example, by point mutation of the DNA encoding the amino acid sequence. Functional equivalents include molecules that bind soluble CLEC12B and comprise all or a portion of the extracellular domains of CLEC12B receptor.

In a particular embodiment, the polypeptide is a peptidomimetic. As used herein, the term “peptidomimetic” refers to a polypeptide designed to mimic a peptide. The polypeptide may be produced by any suitable means, as will be apparent to those of skill in the art. In order to produce sufficient amounts of CLEC12B or functional equivalents thereof for use in accordance with the present invention, expression may conveniently be achieved by culturing under appropriate conditions recombinant host cells containing the polypeptide of the invention. Preferably, the polypeptide is produced by recombinant means, by expression from an encoding nucleic acid molecule. Systems for cloning and expression of a polypeptide in a variety of different host cells are well known. When expressed in recombinant form, the polypeptide is preferably generated by expression from an encoding nucleic acid in a host cell. Any host cell may be used, depending upon the individual requirements of a particular system. Suitable host cells include bacteria mammalian cells, plant cells, yeast and baculovirus systems. Mammalian cell lines available in the art for expression of a heterologous polypeptide include Chinese hamster ovary cells. HeLa cells, baby hamster kidney cells and many others. Bacteria are also preferred hosts for the production of recombinant protein, due to the ease with which bacteria may be manipulated and grown. A common, preferred bacterial host is E coli.

In some embodiments, the CLEC12B antagonist is a short hairpin RNA (shRNA), a small interfering RNA (siRNA) or an antisense oligonucleotide which inhibits the expression of CLEC12B. In a particular embodiment, the CLEC12B antagonist is siRNA. A short hairpin RNA (shRNA) is a sequence of RNA that makes a tight hairpin turn that can be used to silence gene expression via RNA interference. shRNA is generally expressed using a vector introduced into cells, wherein the vector utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs that match the siRNA to which it is bound. Small interfering RNA (siRNA), sometimes known as short interfering RNA or silencing RNA, are a class of 20-25 nucleotide-long double-stranded RNA molecules that play a variety of roles in biology. Most notably, siRNA is involved in the RNA interference (RNAi) pathway whereby the siRNA interferes with the expression of a specific gene. Anti-sense oligonucleotides include anti-sense RNA molecules and anti-sense DNA molecules, would act to directly block the translation of the targeted mRNA by binding thereto and thus preventing protein translation or increasing mRNA degradation, thus decreasing the level of the targeted protein, and thus activity, in a cell. For example, antisense oligonucleotides of at least about 15 bases and complementary to unique regions of the mRNA transcript sequence can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Antisense oligonucleotides, siRNAs, shRNAs of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically mast cells. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

In some embodiments, the CLEC12B antagonist is an endonuclease. In the last few years, staggering advances in sequencing technologies have provided an unprecedentedly detailed overview of the multiple genetic aberrations in cancer. By considerably expanding the list of new potential oncogenes and tumor suppressor genes, these new data strongly emphasize the need of fast and reliable strategies to characterize the normal and pathological function of these genes and assess their role, in particular as driving factors during oncogenesis. As an alternative to more conventional approaches, such as cDNA overexpression or downregulation by RNA interference, the new technologies provide the means to recreate the actual mutations observed in cancer through direct manipulation of the genome. Indeed, natural and engineered nuclease enzymes have attracted considerable attention in the recent years. The mechanism behind endonuclease-based genome inactivating generally requires a first step of DNA single or double strand break, which can then trigger two distinct cellular mechanisms for DNA repair, which can be exploited for DNA inactivating: the errorprone nonhomologous end-joining (NHEJ) and the high-fidelity homology-directed repair (HDR).

In a particular embodiment, the endonuclease is CRISPR-cas. As used herein, the term “CRISPR-cas” has its general meaning in the art and refers to clustered regularly interspaced short palindromic repeats associated which are the segments of prokaryotic DNA containing short repetitions of base sequences.

In some embodiments, the endonuclease is CRISPR-cas9 which is from Streptococcus pyogenes. The CRISPR/Cas9 system has been described in U.S. Pat. No. 8,697,359 B1 and US 2014/0068797. Originally an adaptive immune system in prokaryotes (Barrangou and Marraffini, 2014), CRISPR has been recently engineered into a new powerful tool for genome editing. It has already been successfully used to target important genes in many cell lines and organisms, including human (Mali et al., 2013, Science, Vol. 339 : 823-826), bacteria (Fabre et al., 2014, PLoS Negl. Trop. Dis., Vol. 8:e2671.), zebrafish (Hwang et al., 2013, PLoS One, Vol. 8:e68708.), C. elegans (Hai et al., 2014 Cell Res. doi: 10.1038/cr.2014.11.), bacteria (Fabre et al., 2014, PLoS Negl. Trop. Dis., Vol. 8:e2671.), plants (Mali et al., 2013, Science, Vol. 339 : 823-826), Xenopus tropicalis (Guo et al., 2014, Development, Vol. 141 : 707-714.), yeast (DiCarlo et al., 2013, Nucleic Acids Res., Vol. 41 : 4336-4343.), Drosophila (Gratz et al., 2014 Genetics, doi:10.1534/genetics.113.160713), monkeys (Niu et al., 2014, Cell, Vol. 156 : 836-843.), rabbits (Yang et al., 2014, J. Mol. Cell Biol., Vol. 6 : 97-99.), pigs (Hai et al., 2014, Cell Res. doi: 10.1038/cr.2014.11.), rats (Ma et al., 2014, Cell Res., Vol. 24 : 122-125.) and mice (Mashiko et al., 2014, Dev. Growth Differ. Vol. 56 : 122-129.). Several groups have now taken advantage of this method to introduce single point mutations (deletions or insertions) in a particular target gene, via a single gRNA. Using a pair of gRNA-directed Cas9 nucleases instead, it is also possible to induce large deletions or genomic rearrangements, such as inversions or translocations. A recent exciting development is the use of the dCas9 version of the CRISPR/Cas9 system to target protein domains for transcriptional regulation, epigenetic modification, and microscopic visualization of specific genome loci.

In some embodiments, the endonuclease is CRISPR-Cpf1 which is the more recently characterized CRISPR from Provotella and Francisella 1 (Cpf1) in Zetsche et al. (“Cpf1 is a Single RNA-guided Endonuclease of a Class 2 CRISPR-Cas System (2015); Cell; 163, 1-13).

As used herein the terms “administering” or “administration” refer to the act of injecting or otherwise physically delivering a substance as it exists outside the body (e.g., an antagonist of CELEC12B) into the subject, such as by mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

A “therapeutically effective amount” is intended for a minimal amount of active agent which is necessary to impart therapeutic benefit to a subject. For example, a “therapeutically effective amount” to a subject is such an amount which induces, ameliorates or otherwise causes an improvement in the pathological symptoms, disease progression or physiological conditions associated with or resistance to succumbing to a disorder. It will be understood that the total daily usage of the compounds of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet ofthe subject; the time of administration, route of administration, and rate of excretion of the specific compound employed; the duration of the treatment; drugs used in combination or coincidential with the specific compound employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated.

The present invention relates also to a pharmaceutical composition comprising the antagonist of CLEC12B as described above. The CELC12B antagonists may be combined with pharmaceutically acceptable excipients, and optionally sustained-release matrices, such as biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to one or more of the following agents: solvents such as olive oil, olive oil refined, cottonseed oil, sesame oil, sunflower seed oil, peanut oil, wheat germ oil, soybean oil, jojoba oil, evening primrose oil, coconut oil, palm oil, sweet almond oil, aloe oil, apricot kernel oil, avocado oil, borage oil, hemp seed oil, macadamia nut oil, rose hip oil, pecan oil, hazelnut oil, sasanqua oil, rice bran oil, shea butter, corn oil, camellia oil, grape seed oil, canola oil, castor oil, and combinations thereof, preferably olive oil refined, emulsifiers, suspending agents, decomposers, binding agents, excipients, stabilizing agents, chelating agents, diluents, gelling agents, thickening agent such as beeswax and/or petroleum jelly, preservatives, lubricants, absorption delaying agents, liposomes, antioxidants such as butylhydroxytoluene or butylhydroxyanisole, and the like. The pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Particularly, the pharmaceutical composition is formulated into a topical formulation that can be directly applied to the skin, for example, a skin suffering from vitiligo. The topical formulation suitable for the pharmaceutical composition may be an emulsion, a gel, an ointment, a cream, a patch, an embrocation, an aerosol, a spray, a lotion, a serum, a paste, a foam, or a drop. In one embodiment of this application, the pharmaceutical composition is formulated into an external preparation by admixing the extract according to this application with a base such as those that are well known and commonly used in the art.

A further object of the present invention relates to a method of screening a drug suitable for the treatment of hyperpigmentation disorders comprising i) providing a test compound and ii) determining the ability of said test compound to inhibit the activity of CLEC12B.

Any biological assay well known in the art could be suitable for determining the ability of the test compound to inhibit the activity of CLEC12B. In some embodiments, the assay first comprises determining the ability of the test compound to bind to CLEC12B. In some embodiments, a population of cells is then contacted and activated so as to determine the ability of the test compound to inhibit the activity of CLEC12B. In particular, the effect triggered by the test compound is determined relative to that of a population of immune cells incubated in parallel in the absence of the test compound or in the presence of a control agent either of which is analogous to a negative control condition. The term “control substance”, “control agent”, or “control compound” as used herein refers a molecule that is inert or has no activity relating to an ability to modulate a biological activity or expression. It is to be understood that test compounds capable of inhibiting the activity of CLEC12B, as determined using in vitro methods described herein, are likely to exhibit similar modulatory capacity in applications in vivo. Typically, the test compound is selected from the group consisting of peptides, petptidomimetics, small organic molecules, aptamers or nucleic acids. For example the test compound according to the invention may be selected from a library of compounds previously synthesised, or a library of compounds for which the structure is determined in a database, or from a library of compounds that have been synthesised de novo. In some embodiments, the test compound may be selected form small organic molecules.

The invention will be further illustrated by the following figures and examples. However, these examples and figures should not be interpreted in any way as limiting the scope of the present invention.

### FIGURES

FIG. 1: The downregulation of Clec12B in melanocytes (NHM for normal human melanocyte and MNT1, a melanoma cell line commonly used for studying pigmentation) decreases the transfer of melanin to the keratinocytes in co-culture condition. A. Normalized quantity of melanin in keratinocyte cell lysates cultures with MNT1; B. Normalized quantity of melanin in keratinocyte cell lysates cultures with NHM.

FIG. 2: Silencing of CLEC12B using lenti-shRNA increases the production of melanin in Normal Human Melanocyte (NHM). Quantification of melanin in NHM transduced with control or CLEC12B lentiviral shRNA normalized to protein content.

FIG. 3: Silencing of CLEC12B using lenti-shRNA increases the activity of tyrosinase enzyme as shown by semi-quantification.

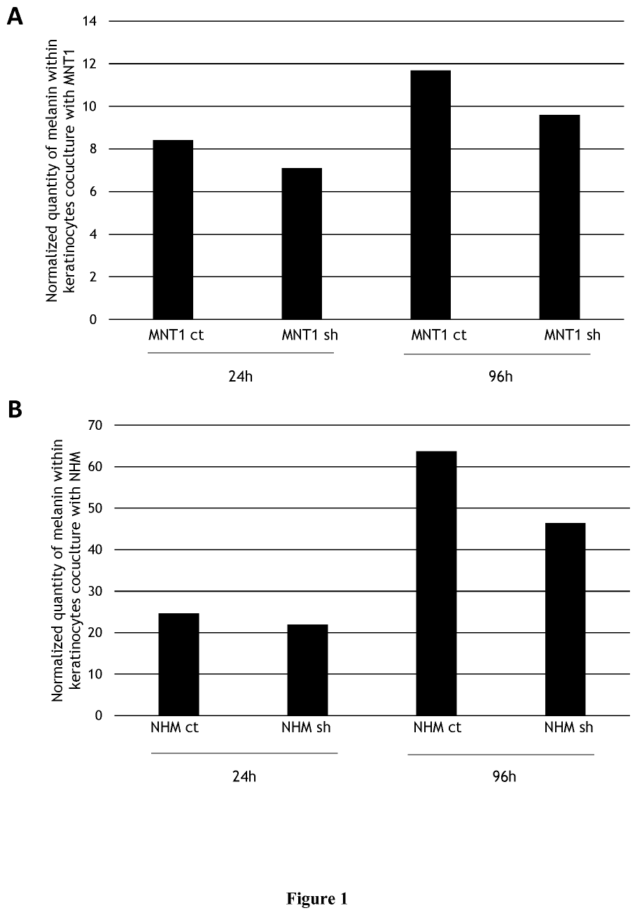

### EXAMPLE

### Material & Methods

### Cell Culture

Normal human melanocytes (NHMs) and normal human keratinocytes (NHKs) were obtained from the foreskin of young children (skin type III or IV) undergoing circumcision. Tissue samples were kindly supplied by the Department of Pediatric Surgery, Lenval Hospital (Dr. Kurzenne, Nice, France). The samples were washed with phosphate buffered saline (PBS) containing 1% Antibiotic/Antimycotic (Gibco, Life Technologies, USA) 3 times for 5 minutes each. After removal of the subcutaneous tissue, tissue was cut into 2×2 mm² pieces.

The foreskin samples were incubated within dispase enzyme (4 U/ml, Roche) for 12-16 h at 4° C. Once the dermis and epidermis of foreskins were separated with forceps, the epidermis was incubated within a trypsin/EDTA solution for 20 minutes at 37° C., the cells dispersed into cell suspensions and filtered by cell strainer (70 μm, Falcon) before final wash with PBS.

NHMs were isolated in MCDB 153 medium (Sigma Aldrich) supplemented with 2% FBS (Fetal Bovin Serum, SV30160-03, Hyclone, USA), 5 μg.ml⁻¹ insulin (Sigma Aldrich), 0.5 μg.ml⁻¹ hydrocortisone (Sigma Aldrich), 16 nM TPA (phorbol 12-myristate 13-acetate , Sigma Aldrich), 1 ng.ml⁻¹ basic fibroblast growth factor (Promega; Madison, Wis.), 15 μg.ml⁻¹ bovine pituitary extract (Gibco, Life Technologies, USA), 10 μM forskolin (Sigma Aldrich), and 20 μg.ml⁻¹ geneticin (Invitrogen) over 2 weeks. Melanocytes between passages 3 and 7 were used.

NHKs were isolated in keratinocyte basal medium 2 (C-20211, Promocell, Heidelberg, Germany) supplemented with human keratinocyte growth supplement (C-39011, HKGS, Cascade Biologics, Calif., USA). Keratinocytes between passages 2 and 5 were used.

MNT-1 human melanoma cells were cultured in DMEM (Gibco, Life Technologies, USA) supplemented with 10% Aim-V medium (Gibco, Life Technologies, USA), 20% fetal bovine serum (Hyclone, Logan, USA), 1 mM Sodium pyruvate (Gibco, Life Technologies, USA) and 0.1 mM nonessential amino acids (Gibco, Life Technologies, USA).

All cells were maintained at 37° C. in a 5% CO2 atmosphere.

### Lentiviral Infection for RNAi Gene Knockdown

CLEC12B siRNA/shRNA/RNAi lentiviral particles (iV004749) and scrambled siRNA GFP Lentiviral particles (LVP015G) were purchased from Applied Biological Materials (Canada).

NHMs were serially passaged and used at passage 3 for viral infection. Cells were plated on 24-well plates at a density of 6×10⁴ cells per well and infected with a multiplicity of infection of 10 in the presence of 8 μg/ml polybrene (Sigma Aldrich). Cells were incubated with virus for 48 hours and polybrene was added during the last 4 hours.The infected cells were selected for stable expression using puromycin at 1 ug/ml. Infected NHMs were cultured in medium 254 supplemented with human melanocyte growth supplement (Cascade Biologics, Calif., USA).

### Co-Culture Experiment

For our coculture model, melanocytes were plated on 6-well plates at a density of 6×10⁴ cells per well. 24 h later, keratinocytes were added to each well (3×10⁵ cells), with an initial seeding ratio of 5:1.

Cocultures were then maintained in in keratinocyte basal medium 2 (C-20211, Promocell, Heidelberg, Germany) supplemented with human keratinocyte growth supplement (C-39011, HKGS, Cascade Biologics, USA).

After 24 or 96 hours of coculture, melanocytes and keratinocytes were separated by differential trypsinization method.

Differential trypsinization was performed as follows: MHNs were initially digested with 0.05% trypsin/EDTA for 2-5 min at 37° C. When MHNs become round and withdraw their dendrites, MHN single-cell suspension was obtained by carefully pipetting the MHN as they are more easily digested out of the substratum than KCs. The KHNs single-cell suspensions were obtained following a second digestion of trypsin. Trypsin is neutralized by medium containing 10% FBS. MHNs and KHNs were pelleted by centrifugation and resuspended in PBS before determination of melanin content and protein content.

### Determination of Melanin Content Melanin Assay

Cell suspensions were centrifuged and pellets photographed before cells were solubilized in 120 μl of 0,5N NaOH at 80° C. for 1 hr to dissolve melanin. Melanin absorbance was measured spectrophotometrically at 405 nm using a plate reader. Melanin production was calculated by normalizing the total melanin values with protein content.

### Results

We have demonstrated that CLEC12B is expressed specifically in the skin by melanocytes. CLEC12B is expressed at the membrane surface of the melanocytes and in the cytoplasm. CLEC12B colocalized with microtubules. It also colocalized with and actine fibers but only when melanocytes are stimulated with αMSH or forskolin or ultraviolet. Using videomicroscopy we have followed the CLEC12B thanks to a lentivirus-GFP/CLEC12B construction and we have observed that after stimulation with αMSH or forskolin, CLEC12B colocalizes with melanosome and interact with the membrane of the keratinocytes. Then, using co-culture experiment with melanocytes and keratinocytes we have showed that decreasing CLEC12B expression significantly reduce the transfer of melanin to the keratinocytes.

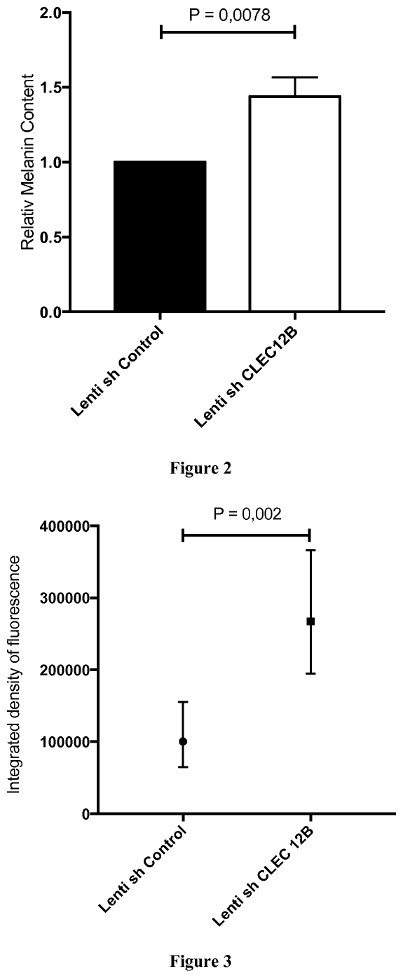

Normal Human Melanocyte (NHM) are transduced with control or CLEC12B lentiviral shRNA. Cells images (data not shown) and cells lysates (data not shown) shown that silencing of CLEC12B using lenti-shRNA increases the production of melanin in NHM. The quantification of melanin in NHM transduced with control or CLEC12B lentiviral shRNA normalized to protein content (FIG. 2).

Silencing of CLEC12B using lenti-shRNA increases the activity of tyrosinase enzyme as shown on FIG. 3 by semi quantification.

Silencing of CLEC12B using lenti-shRNA increases microphthalmia-associated transcription factor (MITF) and melanogenesis gene expression of DCT and Tyrosinase (data not shown).

Taken together these results demonstrate that CLEC12B is specifically expressed in the skin by melanocytes and plays a key role in the transfer of or melanosomes (and thus melanin) to the keratinocytes. Decreasing the expression of CLEC12B or preventing the interaction between CLEC12B with keratinocytes is a specific and effective way to decrease pigmentation in the skin.

### REFERENCES

Throughout this application, various references describe the state of the art to which this invention pertains. The disclosures of these references are hereby incorporated by reference into the present disclosure.

1. A method for treating hyperpigmentary skin disorder in a subject in need thereof comprising a step of administering to said subject a therapeutically effective amount of a CLEC12B antagonist.

2. The method according to claim 1, wherein the CLEC12B antagonist is an antibody.

3. The method according to claim 1, wherein the CLEC12B antagonist is a polypeptide.

4. The method according to claim 3, wherein the the polypeptide is a decoy.

5. The method according to claim 1, wherein the CLEC12B antagonist is a glycomimectic molecule.

6. A method of screening a drug suitable for the treatment of hyperpigmentary skin disorder comprising i) providing a test compound and ii) determining the ability of said test compound to inhibit the activity of CLEC12B.
